(19) United States
(12) Patent Application Publication
(10) Pub. No.: US 2007/0042037 A1
(43) Pub. Date: Feb. 22, 2007

(54) Pharmaceutical composition containing levodopa and carbidopa

(76) Inventor: Alessandro Tagliamonte, Castelnuovo Berardenga (IT)

Correspondence Address: YOUNG & THOMPSON, 745 SOUTH 23RD STREET, 2ND FLOOR, ARLINGTON, VA, 22202, US

(21) Appl. No.: 10/553,388

(22) PCT Filed: Apr. 6, 2004
(86) PCT No.: PCT/EP04/03669

§ 371 Date: Jun. 20, 2006

(30) Foreign Application Priority Data
Apr. 18, 2003 (IT) MI2003A000827

(51) Int. Cl. A61K 9/64; A61K 9/24

(52) U.S. Cl. 424/456; 424/472

(57) ABSTRACT

An oral solid pharmaceutical composition for constant and prolonged release of a levodopa/carbidopa combination, for use in the treatment of Parkinson disease.

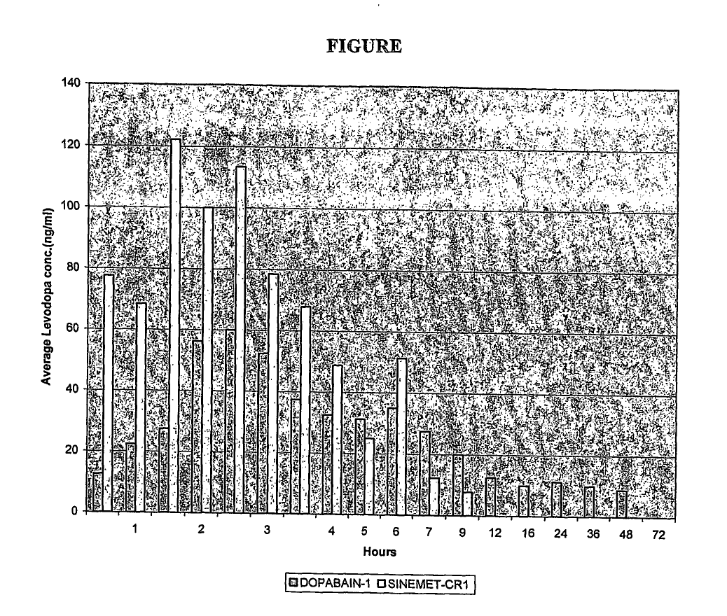

The present invention relates to a prolonged-release oral solid pharmaceutical composition containing the combination levodopa/carbidopa, and the use thereof in the therapy of Parkinson's disease or related pathologies.

### TECHNICAL BACKGROUND

Parkinson's disease is a neurodegenerative disease that involves different areas of the brain, especially the “substantia nigra”, utilizing dopamine as neurotransmitter, causing a slow, progressive disorder of the movements. The main symptoms are bradykinesia, muscular rigidity, resting tremor and postural instability. The diagnosis is usually confirmed by a favourable response to the pharmacological treatment.

The pharmacological therapy is based on the use of selegilins, anticholinergics, amantadines, dopaminergic agonists, ergot alkaloids, levodopa, COMT inhibitors.

Levodopa, upon oral administration, passes the blood-brain barrier and is enzymatically converted to dopamine at the cerebral level.

The choice anti-Parkinson medicament is Sinemet, which contains a combination of levodopa and carbidopa; the latter does not cross the blood-brain barrier, thus reducing the conversion of levodopa to dopamine by peripheral enzymes, thereby increasing the amount of active ingredient available in the central nervous system.

The therapeutic treatment is usually started with Sinemet 25 mg/brain 100 mg (carbidopa/levodopa) ½ tablet a day and after some weeks the dosage is increased until the clinically effective dosage, which is usually 1 tablet 3 or 4 times a day, is reached. Alternatively, treatment with Sinemet CR (sinemet controlled-release) 50 mg/200 mg starts with ½ tablet once a day, and slowly increases until 1 tablet twice a day. The bioavailability of Sinemet CR is approx. 30% lower than that of Sinemet.

Levodopa attains the maximum plasmatic concentration in 1-2 hours and its half-life is 1-3 hours. Therefore, the medicament has to be administered repeatedly during the day and the consequent peaks (Cmax) of plasmatic concentration cause undesired side-effects in the patient. Commercially available carbidopa/levodopa CR (controlled-release) tablets are therapeutically effective over a period of 8 hours, but have various drawbacks and undesired effects, including nausea and vomit, orthostatic hypotension, movement swings, dyskinesias and psychosis.

### DISCLOSURE OF THE INVENTION

The present invention provides a solid oral pharmaceutical composition containing a combination of carbidopa and levodopa, which ensures a steady release of the active ingredient over 24 hours, thus avoiding plasmatic peaks or fluctuations.

The composition according to the invention contains carbidopa and separately levodopa granules coated by an ethylcellulose film, in a 1:4 carbidopa/levodopa weight ratio. The granular mixture is distributed in a suitable pharmaceutical form, preferably in pre-dosed sachets or hard-gelatin capsules.

The dose of active ingredient per unitary dosage can range from 10 to 200 mg for carbidopa and 40 to 800 mg for levodopa, maintaining a 1:4 weight ratio. The daily dosage can be varied depending on the severity of the disease, the general conditions of the patient, and other parameters. Preferably a 250 mg dose, corresponding to 50 mg carbidopa+200 mg levodopa per unitary dosage, is administered once a day.

According to a preferred embodiment, coated granules are prepared from a mixture containing:

| | |
|---|---|
| Active ingredient (levodopa or carbidopa) | 90% |
| Polyethylene glycol (carbowax) | 3% |
| Ethylcellulose | 1.70% |
| Talc | 0.80% |
| Polyvinylpyrrolidone | 4.50% |
| Potassium metabisulfite | q.s. (<0.01%) |

The granulation process comprises mixing the active ingredient with the binders in a solvent, subsequently granulating the mixture, for example by means of a sieve with suitable mesh to obtain the desired particle size distribution, and coating the granules with ethylcellulose.

According to a preferred embodiment, the granules are prepared by separately mixing the active principles with polyethylene glycol and polyvinylpyrrolidone, and granulating the resulting mixture through a sieve, optionally repeating the process through finer sieves. The coating solution, consisting of ethylcellulose and potassium metabisulfite in acetone and denaturated alcohol, is subsequently sprayed onto the granules, with addition of talc to promote flowing of the granulate; finally coated granules are dried to remove any traces of the solvent. After completing the granulation and coating, coated granules are worked up to the final pharmaceutical form, for example distributed in capsules or sachets.

A once a day administration of the pharmaceutical composition according to the invention is particularly advantageous, in that
1) prevents plasma concentration peaks,
2) promotes gradual intestinal absorption,
3 ensures a steady supply of the active principles, according to predetermined amounts and ratios, during 24 hours.

The therapeutic efficacy and the patient's compliance are therefore improved, while minimizing the undesired effects.

Thanks to its pharmacokinetic characteristics, the pharmaceutical composition of the invention is conveniently used for the preventive or therapeutical treatment of Parkinson's disease and related disorders.

The following examples illustrate the invention in greater detail.

### EXAMPLE 1

### Preparation of the bulk LEVODOPA/CARBIDOPA

400 Kg preparation

| | |
|---|---|
| LEVODOPA | 359.960 Kg |
| Carbowax 4000 | 12.000 Kg |
| Ethylcellulose | 6.800 Kg |
| Talc | 3.200 Kg |
| Polyvinylpyrrolidone | 22.000 Kg |
| Potassium metabisulfite | 0.040 Kg |
| Acetone* | |
| Denaturated alcohol* | |
| Demineralised water* | |

100 Kg preparation

| | |
|---|---|
| CARBIDOPA | 89.990 Kg |
| Carbowax 4000 | 3.000 Kg |
| Ethylcellulose | 1.700 Kg |
| Talc | 0.800 Kg |
| Polyvinylpyrrolidone | 4.500 Kg |
| Potassium metabisulfite | 0.010 Kg |
| Acetone* | |
| Denaturated alcohol* | |
| Demineralised water* | |

*solvents used in the process that evaporated off at the end of the process.

Preparation of the Binder Solution

Carbowax 4000 is placed in a stainless steel container fitted with pneumatic stirrer, then polyvinylpyrrolidone is poured, in small amounts, stirring until solubilization.

Granulation

Levodopa and Carbidopa are accurately weighed and passed in the granulator using the above binder solution as aggregant.

The wet granulate is forced through a sieve with 840 micron mesh, dried at 40° C. for 15 hours in forced-air thermostatised drier and subsequently sieved through a 500 and 840 micron mesh sieve. Powder and granules smaller than 500 micron are regranulated with the same procedure as described above, using deionized water as aggregant. After completion of the granulation process, granules are sieved through a 500 and 840 micron sieve.

The resulting granulate (core) is weighed and placed in a stainless steel basket of the coating pan. During the rotation of the coating pan, which takes place at a suitable rate to ensure an effective rotation of the mass (12 rpm), the binder solution is sprayed onto the granules through a sprayer, preventing formation of drops, and the residual powder active ingredient is added. Spraying of the binder and addition of the powder are carried out at alternate intervals in order to adhere a thin layer of the powder to the core granules and provide better evaporation of the solvent (water) present in the binder solution, which is removed by aspiration avoiding the formation of bubbles. Then the wet granulate is passed through a 1200 micron sieve and dried at 40° C. for 15 hours in a forced air thermostatized dryer. After drying, the granulate is sieved again through a 840 and 1200 micron sieve.

Preparation of the Coating Film (Solution)

Acetone and denaturated alcohol are placed in the stainless steel container, then ethylcellulose and potassium metabisulfite are added under continuous stirring, until complete solubilization.

Coating of the Granules

The granulate from the above step is placed in a fluidized bed and kept in suspension by a filtered air stream.

The coating solution is sprayed intermittently through a sprayer so as to prevent formation of drops. Talc is added to promote flowing of the granulate mass. After completion of the process, the granulate is forced through a 1200 micron sieve. The coated granulate is dried at 40° C. for 15 hours in a forced air thermostatized dryer.

Preparation of the Bulk

The dry coated granulate is sieved through a 840 and 1200 micron sieve and the product is collected in a polyethylene double bag and placed in a metal container with hermetic sealing.

### EXAMPLE 2

### Pharmaceutical Preparation in Bulk (Capsules)

The two granulates (Carbidopa and Levodopa, respectively) obtained as in the examples 1 and 2 above, are distributed into hard-gelatin capsules, maintaining a 1:4 weight ratio (carbidopa/levodopa).

Encapsulation was carried out with a machine equipped with two loading trays, double feeder, two separate dosers (one for each feeder) and programmed to fill the hemi-capsules with the established amounts of granulates.

For filling 250 mg capsules (50 mg carbidopa+200 mg levodopa), the granulate dosers are set to weigh about 56.6 mg and 222.3 mg of carbidopa and levodopa, respectively.

### EXAMPLE 3

### Dissolution Test

Six samples of a 250 mg preparation (50+200) were tested using a solution at pH 1.1 (artificial gastric juice) in a continuous-flow dissolution chamber (25 ml/min, 37° C.). The following percentages of release during the 24 hours were obtained (each value is the mean of 6 measurements):

| | |
|---|---|
| | % released |
| In vitro release | average |

| Time | Levodopa | Carbidopa |
|---|---|---|
| 03^{rd} hour | 22% | 26% |
| 06^{rd} hour | 43% | 39% |
| 09^{rd} hour | 59% | 51% |
| 12^{rd} hour | 68% | 63% |
| 24^{rd} hour | 90% | 89% |

The data in the table clearly show that the release of active principles is maintained constant over the entire period of 24 hours.

### EXAMPLE 4

### Bioequivalence Study Between a Formulation of the Invention (50 mg Carbodopa+200 mg Levodopa−Denominated Dopabain) and the Commercial Product Sinemet CR ® (50 mg Carbodopa+200 mg Levodopa), Upon Single Administration

In order to evaluate the plasma levodopa concentration of both formulations after 24 hours a single-dose, randomized, two-period, two-sequence crossover bioavalability study was performed in 10 healthy volunteers.

The levodopa concentration of each subject was monitored up to 72 hours. The results of the study are shown in the Figure.

1. A solid oral pharmaceutical composition containing carbidopa and levodopa in a 1:4 weight ratio, in the form of ethylcellulose-coated granules.

2. A pharmaceutical composition as claimed in claim 1, wherein said granules are included in gelatin capsules or sachets.

3. A composition as claimed in claim 1, containing from 10 to 200 mg carbidopa and 40 to 800 mg levodopa.

4. A composition as claimed in claim 3, containing 50 mg carbidopa and 200 mg levodopa.

5. A composition according to claim 1, containing 90% levodopa and carbidopa, 3% polyethylene glycol, 1.70% ethylcellulose, 0.8% talc, 4.5% polyvinylpyrrolidone, potassium metabisulfite q. s. to 100.

6. A composition as claimed in claim 5, wherein the granules have a mean diameter ranging from 500 to 1200 pm.

7. Method for the preparation of ethylcellulose-coated granules to be used in the treatment of Parkinson's diseases or related pathologies, which comprises admixing an effective amount of carbidopa and levodopa in a 1:4 weight ratio.
